(19) United States
(12) Patent Application Publication
(10) Pub. No.: US 2024/0181240 A1
(43) Pub. Date: Jun. 6, 2024

(54) TRI-TUBE REPOSITIONING SHEATH

(71) Applicant: Boston Scientific Scimed Inc., Maple Grove, MN (US)

(72) Inventors: Qian Liu, Plymouth, MN (US); Victor Kotov, St. Louis Park, MN (US)

(21) Appl. No.: 18/388,131

(22) Filed: Nov. 8, 2023

Related U.S. Application Data
(60) Provisional application No. 63/430,488, filed on Dec. 6, 2022.

(51) Int. Cl. A61M 60/865; A61M 39/06; A61M 60/13; A61M 60/216; A61M 60/419

(52) U.S. Cl. CPC A61M 60/865; A61M 39/06; A61M 60/13; A61M 60/216; A61M 60/419; A61M 2039/062

(57) ABSTRACT

A repositioning sheath for use in percutaneously delivering a medical device into a blood vessel includes an outer body having an outer wall surface and an inner wall surface defining a lumen. The repositioning sheath further includes a first tube defining a first lumen and a second tube defining a second lumen. The first tube is disposed within the lumen of the outer body, and the second tube disposed within the lumen of the outer body.

### CROSS-REFERENCE TO RELATED PATENT APPLICATIONS

This application claims priority to U.S. Provisional Application No. 63/430,488, filed Dec. 6, 2022, which is herein incorporated by reference in its entirety.

### TECHNICAL FIELD

The present disclosure relates to a repositioning sheath for use in delivering medical devices. More particularly, the present disclosure relates to a repositioning sheath having at least two tubes disposed therein for receiving at least two medical devices.

### BACKGROUND

In various procedures for delivering intravascular medical devices, an introducer sheath is inserted into a blood vessel of a patient, for example a femoral artery, and medical devices are inserted into the introducer sheath for introduction into the patient's vasculature. In various instances, the medical devices include catheters or other devices such as a blood pump. After delivery of the medical devices, it may be desired to replace the introducer sheath with a repositioning sheath that allows for the repositioning of the delivered medical devices but is smaller in size than the introducer sheath. The replacement of the introducer sheath with a repositioning sheath may increase blood flow and thereby reduce the possibility of ischemia within the blood vessel. In these instances, it may be desired for the repositioning sheath to receive at least two medical devices simultaneously. There is a need for an improved repositioning sheath that may receive two medical devices simultaneously.

### SUMMARY

In Example 1, a repositioning sheath for use in percutaneously delivering a medical device into a blood vessel includes an outer body having an outer wall surface and an inner wall surface defining a lumen, a first tube defining a first lumen, the first tube disposed within the lumen of the outer body, and a second tube defining a second lumen, the second tube disposed within the lumen of the outer body.

In Example 2, the repositioning sheath of Example 1 further includes wherein the first tube is radially spaced from the inner wall surface of the outer body.

In Example 3, the repositioning sheath of Example 1 or Example 2 further includes wherein the second tube is radially spaced from the inner wall surface of the outer body.

In Example 4, the repositioning sheath of any one of Examples 1-3, wherein the second tube and the first tube are arranged such that the first tube and the second tube are in contact with one another.

In Example 5, the repositioning sheath of any one of Examples 1˜4 further includes wherein the first tube has an inner diameter of approximately 0.094 inches and an outer body of approximately 0.110 inches, and wherein the second tube has an inner diameter of approximately 0.039 inches and an outer diameter of approximately 0.043 inches.

In Example 6, the repositioning sheath of Example 5 further includes wherein the sheath has a distal end opposite a proximal end and wherein the distal end of the sheath comprises a tapered distal tip having a length of approximately 3 cm.

In Example 7, the repositioning sheath of any one of Examples 1-6 wherein the tapered distal tip is composed of a 40 durometer Polyether-Block-Amide.

In Example 8, a delivery system for positioning at least one medical device into a blood vessel includes a repositioning sheath having an outer body extending between a proximal end and a distal end, the outer body defining a lumen and the repositioning sheath configured for insertion into the blood vessel, the repositioning sheath further including a first tube defining a first lumen, the first tube disposed within the lumen of the outer body, and a second tube defining a second lumen, the second tube disposed within the lumen of the outer body. The delivery system further includes a hub engaged with the proximal end of the sheath.

In Example 9, the delivery system of Example 8 further includes wherein the first tube is radially spaced from an inner wall surface of the outer body, and optionally, wherein the second tube is radially spaced from the inner wall surface of the outer body.

In Example 10, the delivery system of Example 8 or Example 9 further includes wherein the hub is thermomoulded with the proximal end of the repositioning sheath such that axial and radial positioning of the first tube and the second tube is fixed at the proximal end of the repositioning sheath.

In Example 11, the delivery system of any one of Examples 8-10 further includes wherein the distal end of the repositioning sheath comprises a tapered distal tip that is formed of molded polymer material such that the axial and radial positioning of the first tube and the second tube is fixed at the distal end.

In Example 12, the delivery system of any one of Examples 8-11 further includes wherein the first tube has an inner diameter of approximately 0.094 inches and an outer diameter of approximately 0.110 inches.

In Example 13, the delivery system of any one of Examples 8-12 further includes wherein the second tube has an inner diameter of approximately 0.039 inches and an outer diameter of approximately 0.043 inches.

In Example 14, the delivery system of any one of Examples 8-13 further includes wherein the tapered distal tip is composed of a 50 durometer Polyether-Block-Amide.

In Example 15, the delivery system of any one of Examples 8-14 further includes wherein the outer body of the repositioning sheath is composed of 55 durometer Polyether-Block-Amide.

In Example 16, a repositioning sheath for use with a percutaneous intravascular blood pump includes an outer body having a proximal end and a distal end opposite the proximal end, and a lumen extending between the proximal end and the distal end, and the outer body having an outer wall surface and an inner wall surface, a first tube defining a first lumen, the first tube disposed within the lumen of the outer body, a second tube defining a second lumen, the second tube disposed within the lumen of the outer body, and wherein the first tube is radially spaced from the inner wall surface of the outer body and the second tube is radially spaced from the inner wall surface of the outer body.

In Example 17, the repositioning sheath of Example 16 further includes wherein the first tube and the second tube are arranged such that the first tube and the second tube are in contact with one another.

In Example 18, the repositioning sheath of Example 16 further includes wherein the outer body of the repositioning sheath has an inner diameter of approximately 0.166 inches and an outer diameter of approximately 0.206 inches.

In Example 19, the repositioning sheath of Example 16 further includes wherein the first tube has an inner diameter of approximately 0.094 inches and an outer diameter of approximately 0.110 inches and wherein the second tube has an inner diameter of approximately 0.039 inches and an outer diameter of approximately 0.043 inches.

In Example 20, the repositioning sheath of Example 16 further includes wherein the distal end of the repositioning sheath comprises a tapered distal tip having a length of approximately 3 cm.

In Example 21, the repositioning sheath of Example 20 further includes wherein the tapered distal tip is composed of a 40 durometer Polyether-Block-Amide.

In Example 22, the repositioning sheath of Example 16 further includes wherein the outer body of the repositioning sheath is composed of 55 durometer Polyether-Block-Amide.

In Example 23, a delivery system for positioning at least one medical device into a blood vessel includes a repositioning sheath having an outer body extending between a proximal end and a distal end, the outer body defining a lumen and the repositioning sheath configured for insertion into the blood vessel, the repositioning sheath further including a first tube defining a first lumen, the first tube disposed within the lumen of the outer body, a second tube defining a second lumen, the second tube disposed within the lumen of the outer body, and wherein the first tube is radially spaced from the inner wall surface of the outer body and the second tube is radially spaced from the inner wall surface of the outer body. The delivery system further includes a hemostasis valve hub engaged with the proximal end of the repositioning sheath.

In Example 24, the delivery system of Example 23 further includes wherein the hub is thermomoulded with the proximal end of the repositioning sheath such that the axial and radial positioning of the first tube and the second tube is fixed at the proximal end of the repositioning sheath.

In Example 25, the delivery system of Example 24 further includes wherein the distal end of the repositioning sheath comprises a tapered distal tip that is formed of molded polymer material such that the axial and radial positioning of the first tube and the second tube is fixed at the distal end.

In Example 26, the delivery system of Example 24 further includes wherein the tapered distal tip has a length of approximately 3 cm.

In Example 27, the delivery system of Example 23 further includes wherein the outer body of the repositioning sheath has an inner diameter of approximately 0.166 inches and an outer diameter of approximately 0.206 inches.

In Example 28, the delivery system of Example 23 further includes wherein the first tube has an inner diameter of approximately 0.094 inches and an outer diameter of approximately 0.110 inches.

In Example 29, the delivery system of Example 23 further includes wherein the second tube has an inner diameter of approximately 0.039 inches and an outer diameter of approximately 0.043 inches.

In Example 30, the delivery system of Example 23 further includes wherein the tapered distal tip is composed of a 40 durometer Polyether-Block-Amide.

In Example 31, the delivery system of Example 23 further includes wherein the outer body of the repositioning sheath is composed of 55 durometer Polyether-Block-Amide.

In Example 32, a delivery system for positioning at least one medical device into a blood vessel including a repositioning sheath having an outer body extending between a proximal end and a distal end, the outer body defining a lumen and the repositioning sheath configured for insertion into the blood vessel, the repositioning sheath including a first tube defining a first lumen, the first tube disposed within the lumen of the outer body, a second tube defining a second lumen, the second tube disposed within the lumen of the outer body, and wherein the repositioning sheath has an inner diameter of approximately 0.166 inches, the first tube has an outer diameter of approximately 0.110 inches, and the second tube has an outer diameter of approximately 0.39 inches, such that the first tube is radially spaced from the inner wall surface of the outer body and the second tube is radially spaced from the inner wall surface of the outer body. The delivery system further includes a hemostasis valve hub engaged with the proximal end of the repositioning sheath.

In Example 33, the delivery system of Example 32 further includes wherein the hub is thermomoulded with the proximal end of the repositioning sheath such that the axial and radial positioning of the first tube and the second tube is fixed at the proximal end of the repositioning sheath.

In Example 34, the delivery system of Example 33 further includes wherein the distal end of the repositioning sheath comprises a tapered distal tip that is formed of molded polymer material such that the axial and radial positioning of the first tube and the second tube is fixed at the distal end.

In Example 35, the delivery system of Example 34 further includes wherein the tapered distal tip has a length of approximately 3 cm.

### BRIEF DESCRIPTION OF THE DRAWINGS

FIG. 1 illustrates a side view of an introducer sheath extending into a blood vessel, in accordance with elements of the present disclosure.

FIG. 2 illustrates a cross sectional view of a medical device positioned within a blood vessel, in accordance with embodiments of the present disclosure.

FIG. 3 illustrates a side perspective view of a repositioning sheath attached to a hub, in accordance with embodiments of the present disclosure.

FIG. 4 illustrates a cross sectional view of the repositioning sheath of FIG. 3.

### DETAILED DESCRIPTION

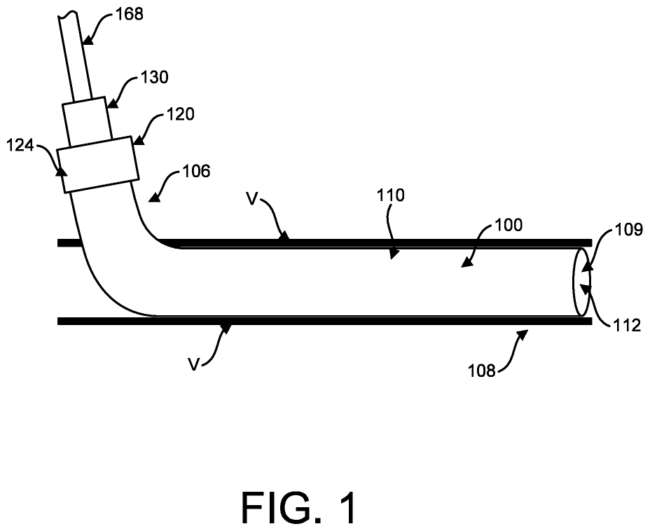

FIG. 1 illustrates a side cross sectional view of a blood vessel V with an introducer sheath 100, inserted at least partially into the blood vessel V. In some embodiments, the introducer sheath 100 is used for facilitating the passage of various relatively large medical devices, such as a blood pump, as will be described further herein, through the introducer sheath 100 and into the blood vessel V. Hence, the introducer sheath 100 may be referred to as a large bore introducer sheath. The introducer sheath 100 comprises a proximal end 106 and a distal end 108 that is opposite the proximal end 106. The introducer sheath 100 includes a proximal opening adjacent the proximal end 106 and a distal opening 109 adjacent the distal end 108. A body portion 110 of the introducer sheath 100 extends between the proximal end 106 and the distal end 108, and the body portion 110 defines a lumen 112 of the introducer sheath 100. The introducer sheath 100 may be formed by various polymeric or metallic materials. In further embodiments, the introducer sheath 100 may comprise an additional surface coating. The surface coating may include, but is not limited to, silicone, PET, or any other applicable polymer.

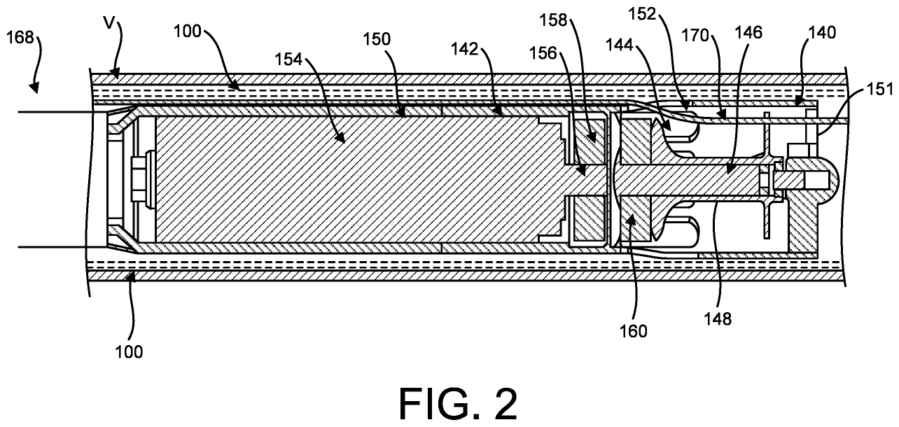

A hub 120 is commonly included at the proximal end 106 and over the proximal opening 107 of the introducer sheath 100. The hub 120, also referred to herein as a hemostasis valve hub, is configured for hemostasis, i.e., to prevent blood from leaking out of the introducer sheath 100 during use. More specifically, a medical device, for example a catheter 168, may be inserted through the hub 120 and the introducer sheath 100 and into the blood vessel V, and the hub 120 may maintain hemostasis between the catheter 168, the introducer sheath 100, and the external surroundings. In some embodiments, the catheter 168 may couple to a medical device such as the blood pump 150 shown in FIG. 2. After insertion of the catheter 168, fixation of the axial and radial position of the catheter 168 may be desired to ensure that the catheter 168 (and any coupled medical device) is in the proper position during use. Further, in some instances, it may also be desired for the operator to reposition the catheter 168 (and any coupled medical device) after insertion. As such, in some embodiments, the hub 120 may comprise a tightening port 130 composed of several components within the hub 120 that provides for fixation of the catheter 168 with respect to the hub 120 and blood vessel V. However, in other embodiments, the tightening port 130 may not be incorporated.

FIG. 2 illustrates a cross-sectional view of the introducer sheath 100 of FIG. 1 after insertion of a medical device, illustratively a blood pump 150, into the introducer sheath 100. As noted above, a catheter such as the catheter 168 may be coupled to the proximal end of the blood pump 150 and extend outside the blood vessel V and introducer sheath 100. The blood pump 150 generally includes an impeller assembly housing 140 and a motor housing 142. In some embodiments, the impeller assembly housing 140 and the motor housing 142 may be integrally or monolithically constructed. The impeller assembly housing 140 carries an impeller assembly 144 therein. The impeller assembly 144 includes an impeller shaft 146 and an impeller 148 that rotates relative to the impeller assembly housing 140 to drive blood through the blood pump 150. More specifically, the impeller 148 causes blood to flow from a blood inlet 151 formed on the impeller assembly housing 140, through the impeller assembly housing 140, and out of a blood outlet 152 formed on the impeller assembly housing 140. In some embodiments the impeller shaft 146 and the impeller 148 may be integrally formed, and in other embodiments the impeller shaft 146 and the impeller 148 may be separate components. As shown in FIG. 2, the inlet 151 may be formed on an end portion of the impeller assembly housing 140, and the outlet 152 may be formed on a side portion of the impeller assembly housing 140. In other embodiments, the inlet 151 and/or the outlet 152 may be formed on other portions of the impeller assembly housing 140. In some embodiments, the impeller assembly housing 140 may couple to a distally extending cannula, and the cannula may receive and deliver blood to the inlet 151.

With continued reference to FIG. 2, the motor housing 142 carries a motor 154, and the motor 154 is configured to rotatably drive the impeller 148 relative to the impeller assembly housing 140. In the illustrated embodiment, the motor 154 rotates a drive shaft 156, which is coupled to a driving magnet 158. Rotation of the driving magnet 158 causes rotation of a driven magnet 160, which is connected to the impeller assembly housing 140. More specifically, in embodiments incorporating the impeller shaft 146, the impeller shaft 146 and the impeller 148 are configured to rotate with the driven magnet 160. In other embodiments, the motor 154 may couple to the impeller assembly housing 140 via other components. While the introducer sheath 100 is illustrated above with the use of the blood pump 150, various other medical devices may be used in conjunction with the introducer sheath 100 and the hemostasis valve hub 120.

While the introducer sheath 100 may be used for the initial delivery of the medical devices into the blood vessel V, it may be desired to replace the introducer sheath 100 with a smaller sheath after delivery of the devices to increase blood flow and thereby reduce chances of ischemia or blood flow blockage within the blood vessel V. In these instances, the introducer sheath 100 may be removed and replaced with a repositioning sheath having a smaller size, and more particularly smaller diameter, than the introducer sheath 100. As will be described further herein, the repositioning sheath may have the hub 120 coupled therewith. The delivered medical devices, or portions thereof, fit within the repositioning sheath, and the repositioning sheath allows for positioning or repositioning of one or more of the delivered medical devices within a patient's body.

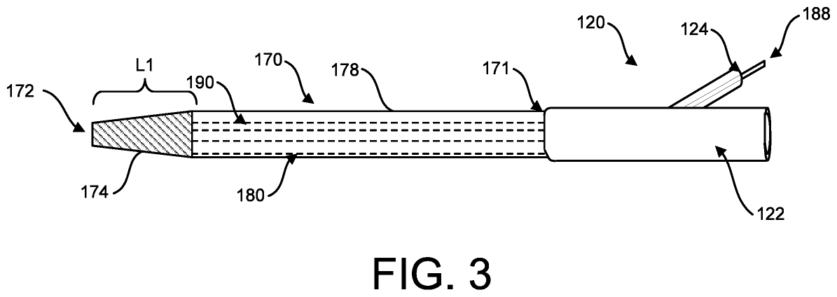

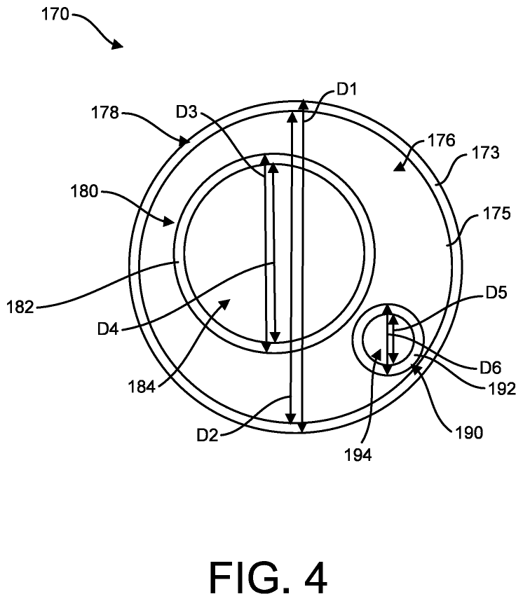

For example, FIG. 3 illustrates a side view of a repositioning sheath 170 having a proximal end 171 coupled with the hub 120. The hub 120 may have a first arm 122 and a second arm 124 such that at least two medical devices may extend through the hub 120 simultaneously. In these instances, the devices may be received through the repositioning sheath 170 and also within the hub 120. As illustrated in FIG. 3, the repositioning sheath 170 has a proximal end 171 that is coupled with the hub 120, and a distal end 172 defined by a tapered distal tip 174. The repositioning sheath 170 has an outer body 178 extending between the proximal end 171 and the distal end 172. The outer body 178 is defined by an outer surface 173 and an inner surface 175, as is shown in the cross-sectional view of FIG. 4. The outer body 178 may be composed of a polymeric material including, but not limited to, silicone, polyether-block-amide (PEBAX®), polyurethane, Hytrel®, or various other thermoformed or thermoset polymers. The material of the outer body 178 may also have varying levels of stiffness. For example, in some instances, the outer body 178 may be composed of 55 durometer PEBAX®. However, various other durometer values may characterize the material composed by the outer body 178 and the above-described examples are not meant to be limiting.

As shown in phantom lines in FIG. 3, a first tube 180 and a second tube 190 extend through the repositioning sheath 170. Lumens defined by the first arm 122 and the second arm 124 of the hub 120 may be fluidly connected with the first and second tubes 180, 190, respectively. Such an arrangement allows medical devices extending through the first and second tubes 180, 190 to extend through the first arm 122 and the second arm 124 of the hub 120, respectively. For example, medical devices extending through the first arm 122 may be inserted into the first tube 180 and medical devices extending through the second arm 124 may be inserted into the second tube 190. Even further, the first tube 180 and the second tube 190 extend through the first and second arms 122, 124 independently from one another. In other words, the first tube 180 and the second tube 190 can receive devices therethrough without the devices being in contact with one another. A medical device, such as a catheter, extending within the first tube 180 may extend into the first arm 122 while another medical device, such as a stylet, inserted into second arm 124 may extend through second tube 190. Due to the arrangement of the first tube 180 and the second tube 190, the devices received therein, such as the catheter and the stylet, fail to be in contact with one another during operation of the repositioning sheath 170. As illustrated in FIG. 3, the hub 120 and the repositioning sheath 170 may be configured for receiving a plug 188 or a stylet which may be received within the second arm 124 and the second tube 190 when there is not a medical device received within the second arm 124 and the second tube 190. This reduces the possibility that blood will leak out of the hub 120 through the second arm 124 while the hub 120 and the repositioning sheath 170 are being used.

In some instances, the hub 120 is thermomoulded onto the proximal end 171. In further examples, the hub 120 is glued onto the proximal end 171. In this way, the proximal end 171 is molded such that first tube 180 and the second tube 190 arranged within the repositioning sheath 170 are secured with one another at the proximal end 171 and held in place axially and radially, as will be described further herein. In other embodiments, the first tube 180 and the second tube 190 may be molded within the repositioning sheath 170 prior to coupling with the hub 120. Thus, even without the incorporation of the hub 120, the first tube 180 and the second tube 190 may be axially and radially secured at the proximal end 171 of the repositioning sheath 170. Further, in some embodiments, the distal tip 174 is formed of polyamide that has been melted or thermomoulded such that the distal tip 174 is molded and solidified. In this way, the first tube 180 and the second tube 190 may be molded within the repositioning sheath 170 at the distal end 172, as well. Thus, the axial and radial positioning of the first tube 180 and the second tube 190 may be secured at the distal end 172 of the repositioning sheath 170.

Further, the distal tip 174 may be formed of a radiopaque material such that when the repositioning sheath 170 is inserted into the patient, the operator may monitor the positioning of the distal tip 174 using various imaging processes. With continued reference to FIG. 3, the distal tip 174 is defined by a length L1. In some embodiments, the length L1 may have a value that ranges between approximately 1 cm and approximately 6 cm. For example, in some instances, the length L1 has a value of approximately 3 cm. It may be desired to have a longer length L1 as the tapered design of the distal tip 174 may ease the transition of the repositioning sheath 170 into the blood vessel V due to the tapered diameter. In other words, the tapered design of the distal tip 174 allows for more blood flow around the repositioning sheath 170 at the distal end 172 of the repositioning sheath 170 to increase the ease with which blood can flow around the repositioning sheath 170.

The properties and arrangement of the first tube 180, the second tube 190 and the repositioning sheath 170 will be described further with reference to the cross-sectional view of FIG. 4. As illustrated, the outer body 178 of the repositioning sheath defines a lumen 176 and the lumen 176 extends between the proximal end 171 and the distal end 172. The outer body 178 comprises the outer surface 173 which is defined by an outer diameter D1. The outer diameter D1 may have a value ranging between approximately 3 mm to approximately 7 mm. For example, in some instances, the value of the diameter D1 is approximately 5.2 mm. Further, the outer body 178 may comprise the inner surface 175 that is defined by an inner diameter D2. The inner diameter D2 may have a value ranging between approximately 2 mm and approximately 6 mm. For example, in some instances, the inner diameter D2 may have a value of 4.2 mm.

The first tube 180 and the second tube 190 are arranged within the lumen 176 of the outer body 178. The first tube 180 is defined by an outer body 182 that further defines a lumen 184. Further, the first tube 180 is defined by an outer diameter D3 and an inner diameter D4. In some embodiments, the value of the outer diameter D3 is between approximately 0.082 inches (˜2 mm) and approximately 0.230 inches (˜5.8 mm). For example, in some instances, the value of the outer diameter D3 is approximately 0.110 inches (˜2.8 mm). In further embodiments, the value of the inner diameter D4 is between approximately 0.066 inches (˜1.7 mm) and approximately 0.214 inches (˜5.4 mm). For example, in some instances, the value of the inner diameter D4 is approximately 0.094 inches (˜2.4 mm). Further, in some instances and as illustrated in the cross-sectional view of FIG. 4, the first tube 180 may remain radially spaced from the inner surface 175 of the outer body 178 of the repositioning sheath 170. This is at least in part due to the outer diameter D3 being less than the value of inner diameter D2. However, in other embodiments, the first tube 180 and the second tube 190 may be configured such that the first tube 180 and the second tube 190 are in contact with the inner surface 175 of the outer body 178. In further embodiments, only one of the first tube 180 and the second tube 190 may be in contact with the inner surface 175 of the outer body 178. For example, in some instances, the first tube 180 may be radially spaced from the inner surface 175 of the outer body 178 while the second tube 190 is in contact with the inner surface 175. In other instances, the first tube 180 may be in contact with the inner surface 175 of the outer body 178 while the second tube 190 remains radially spaced from the inner surface 175 of the outer body 178. Additionally, the first tube 180 and the second tube 190 may be arranged such that the tubes 180, 190 are in contact with one another. In other embodiments, the first tube 180 is spaced from the second tube 190. However, various other arrangements of the first tube 180 and the second tube 190 may be used.

With continued reference to FIG. 4, the second tube 190 is also illustrated as arranged within the lumen 176 of the repositioning sheath 170. The second tube 190 comprises an outer body 192 that defines a lumen 194 extending therethrough. Additionally, the second tube 190 comprises an inner diameter D5 and an outer diameter D6. The value of the inner diameter D5 may range from approximately 0.018 inches (˜. 5 mm) to approximately 0.1 inches (˜2.5 mm). For example, in some instances, the value of the inner diameter D5 is approximately 0.039 inches (˜1 mm). The value of the outer diameter D6 may range from approximately 0.021 inches (˜. 5 mm) to approximately 0.11 inches (˜2.8 mm). For example, in some instances, the value of the outer diameter D6 is approximately 0.043 inches (˜1 mm).

Similar to that described with reference to the first tube 180, the second tube 190 is arranged such that it may be radially spaced from the inner surface 175 of the outer body 178 of the repositioning sheath 170. Even further, the second tube 190 is illustrated as radially spaced from the first tube 180. In this way, the outer body 182 of the first tube 180 and the outer body 192 of the second tube 190 may not be in contact with one another or in contact with the inner surface 175 of the outer body 178 of the repositioning sheath 170. Thus, while the repositioning sheath 170, the first tube 180 and the second tube 190 are molded with one another and radially and axially secured at the distal end 172 and the proximal end 171 of the repositioning sheath 170. The first tube 180 and the second tube 190 may remain spaced from one another and the inner surface 175 of the outer body 178 when extending between the proximal end 171 and the distal end 172. Due to secured positioning of the first tube 180 and the second tube 190 at the distal end 172 and the proximal end 171, shifting or sliding of the first and second tubes 180, 190 within the outer body 178 is reduced. In other embodiments, the first tube 180 and second tube 190 may be in contact with each other. In other embodiments, the first tube 180 and/or the second tube 190 may be in contact with the inner surface 175 of the outer body 178 of the repositioning sheath 170.

As described herein, the first tube 180 and the second tube 190 are configured for receiving medical devices extending therethrough. For example, the first tube 180 may be configured for receiving a catheter, a blood pump, a guidewire, a guide catheter, or a small sheath. Further, the second tube 190 may be configured for receiving a guidewire, a stylet, a plug, contrast injection, drug injection or other mechanism of flushing with heparin or saline. However, the above listed medical devices and substances are provided merely as examples and further medical devices or substances may be used with the first tube 180 and/or the second tube 190.

The above-described configuration of the first tube 180 and the second tube 190 being arranged within the repositioning sheath 170 while the repositioning sheath 170 is coupled with the hub 120 allows for two separate medical devices to be extended into or through the hub 120 and into or through the repositioning sheath 170 while remaining separated from one another. This may reduce the potential of the medical devices damaging one another or engaging with one another during the insertion, repositioning, and/or the removal of the medical devices.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the above-described features.

We claim:

1. A repositioning sheath for use with a percutaneous intravascular blood pump, the repositioning sheath comprising:
an outer body having a proximal end and a distal end opposite the proximal end, the outer body defining a lumen extending between the proximal end and the distal end, the outer body having an outer wall surface and an inner wall surface;
a first tube defining a first lumen, the first tube disposed within the lumen of the outer body; and
a second tube defining a second lumen, the second tube disposed within the lumen of the outer body,
wherein the first tube is radially spaced from the inner wall surface of the outer body and the second tube is radially spaced from the inner wall surface of the outer body.

2. The repositioning sheath of claim 1, wherein the first tube and the second tube are arranged such that the first tube and the second tube are in contact with one another.

3. The repositioning sheath of claim 1, wherein the first tube has a first inner diameter, wherein the second tube has a second inner diameter, wherein the first inner diameter is larger than the second inner diameter.

4. The repositioning sheath of claim 3, wherein the first inner diameter is approximately 2.4 mm, wherein the second inner diameter is approximately 1 mm.

5. The repositioning sheath of claim 1, wherein the distal end of the repositioning sheath comprises a tapered distal tip.

6. The repositioning sheath of claim 5, wherein the tapered distal tip is composed of a 40 durometer Polyether-Block-Amide.

7. The repositioning sheath of claim 1, wherein the outer body of the repositioning sheath is composed of 55 durometer Polyether-Block-Amide.

8. A delivery system for positioning at least one medical device into a blood vessel, the delivery system comprising:
a repositioning sheath having an outer body extending between a proximal end and a distal end, the outer body defining a lumen and the repositioning sheath configured for insertion into the blood vessel, the repositioning sheath further comprising:
a first tube defining a first lumen, the first tube disposed within the lumen of the outer body, and
a second tube defining a second lumen, the second tube disposed within the lumen of the outer body, wherein the first tube is radially spaced from the inner wall surface of the outer body and the second tube is radially spaced from the inner wall surface of the outer body; and
a hemostasis valve hub engaged with the proximal end of the repositioning sheath.

9. The delivery system of claim 8, wherein the hub is thermomoulded with the proximal end of the repositioning sheath such that the axial and radial positioning of the first tube and the second tube is fixed at the proximal end of the repositioning sheath.

10. The delivery system of claim 9, wherein the distal end of the repositioning sheath comprises a tapered distal tip that is formed of molded polymer material such that the axial and radial positioning of the first tube and the second tube is fixed at the distal end.

11. The delivery system of claim 10, wherein the tapered distal tip has a length of approximately 3 cm.

12. The delivery system of claim 10, wherein the tapered distal tip is composed of a 40 durometer Polyether-Block-Amide.

13. The sheath of claim 8, wherein the first tube has a first inner diameter, wherein the second tube has a second inner diameter, wherein the first inner diameter is larger than the second inner diameter.

14. The delivery system of claim 13, wherein the first inner diameter is approximately 2.4 mm.

15. The delivery system of claim 14, wherein the second inner diameter is approximately 1 mm.

16. The delivery system of claim 8, wherein the outer body of the repositioning sheath is composed of 55 durometer Polyether-Block-Amide.

17. A delivery system for positioning at least one medical device into a blood vessel, the delivery system comprising:
a repositioning sheath having an outer body extending between a proximal end and a distal end, the outer body defining a lumen and the repositioning sheath configured for insertion into the blood vessel, the repositioning sheath further comprising:
a first tube defining a first lumen, the first tube disposed within the lumen of the outer body, and
a second tube defining a second lumen, the second tube disposed within the lumen of the outer body,
wherein the first tube is radially spaced from an inner wall surface of the outer body and the second tube is radially spaced from the inner wall surface of the outer body; and
a hemostasis valve hub engaged with the proximal end of the repositioning sheath.

18. The delivery system of claim 17, wherein the hub is thermomoulded with the proximal end of the repositioning sheath such that the axial and radial positioning of the first tube and the second tube is fixed at the proximal end of the repositioning sheath.

19. The delivery system of claim 18, wherein the distal end of the repositioning sheath comprises a tapered distal tip that is formed of molded polymer material such that the axial and radial positioning of the first tube and the second tube is fixed at the distal end.

20. The delivery system of claim 19, wherein the tapered distal tip has a length of approximately 3 cm.
